(19) United States
(12) Patent Application Publication
(10) Pub. No.: US 2019/0308000 A1
(43) Pub. Date: Oct. 10, 2019

(54) Dual-Lumen Drug Reservoir Fill and Withdrawal Devices and Methods

(71) Applicant: Cylerus, Inc., Melbourne, FL (US)

(72) Inventor: Stephen R. HANSON, Edmonds, WA (US)

(21) Appl. No.: 16/373,977

(22) Filed: Apr. 3, 2019

Related U.S. Application Data
(60) Provisional application No. 62/652,430, filed on Apr. 4, 2018.

(51) Int. Cl. A61M 31/00; A61M 5/142; A61F 2/06

(52) U.S. Cl. CPC A61M 31/002; A61F 2250/0067; A61F 2/064; A61M 5/14276

(57) ABSTRACT

Dual-lumen devices for delivery of drugs to a drug reservoir, blood vessel or vascular graft and methods of using the same are provided. In one aspect, devices comprising a dual-lumen needle and dual-lumen catheter are provided for filling and removing drug formulations from a drug reservoir are provided. The drug reservoir can optionally be connected to or associated with a vascular graft or blood vessel for controlled delivery of the drug.

All references cited herein, including but not limited to patents and patent applications, are incorporated by reference in their entirety.

### CROSS-REFERENCE TO RELATED APPLICATIONS

This application claims priority to and the benefit of U.S. Provisional Application No. 62/652,430 filed Apr. 4, 2018, which is hereby incorporated by reference in its entirety.

### BACKGROUND

Local drug delivery in the body remains a challenging problem. While efficacious drugs have been identified and characterized, controlled delivery of such drugs at a sufficient concentration for a sufficient amount of time while avoiding detrimental systemic side effects remains elusive.

Blood conduits may be constructed of either native arteries or veins, or synthetic materials such as expanded polytetrafluoroethylene (ePTFE) graft material, all of which are frequently used in vascular surgery. Vascular grafts are commonly employed for the creation of arteriovenous (A-V) access used as needle insertion sites to enable blood removal and return for hemodialysis that is performed 2-3 times per week in patients with end stage renal disease (ESRD). More than 75,000 new hemodialysis grafts are placed in the U.S. each year and costs for creating and maintaining these grafts exceed $1 billion annually.

Vascular grafts are also indicated in the treatment of peripheral vascular disease (PVD) that is the result of atherosclerosis causing arterial obstruction with pain and cramping in the legs, especially below the knee where vessels are smaller. A blood conduit, such as ePTFE or native vein, is often used to bypass the obstructed artery. The durability and long-term patency of blood conduits used to replace diseased arteries in PVD are substantially better than results with ePTFE grafts used to provide chronic blood access for hemodialysis.

Over 80% of arteriovenous access grafts and 20% of peripheral arterial bypass grafts will fail or become dysfunctional within one year after implantation resulting in considerable patient morbidity and substantial costs to the healthcare system. Dialysis access graft failure is often due to neointimal hyperplasia (i.e., obstructive tissue ingrowth) at the venous outflow tract that is caused by mechanical injury to blood vessels. While drugs that inhibit vascular neointimal hyperplasia in these settings are available, delivery of these drugs to the site of injury, at a safe yet effective dose, for a sufficient period of time, has been challenging.

U.S. Pat. No. 5,399,352 ('352 patent) is directed to placing drug(s) in an external cuff-reservoir for delivery of drug(s) across the wall of the vascular graft to the graft luminal surface. Tubing may be attached to permit refilling or changing of the drug. The '352 patent also refers to use of a device such as a pump to create positive pressure and provide constant controlled drug delivery. However, if the size of the pores in the graft material vary, drug delivery will be non-uniform, and neointimal hyperplasia will not be adequately inhibited. U.S. Pat. No. 8,721,711 ('711 patent) adds a microporous membrane within the graft cuff-reservoir where the pore size can be selected to provide more uniform drug delivery.

U.S. Pat. No. 8,808,255 ('255 patent) provides for a drug delivery cuff-reservoir that can be removed or repositioned.

However, the drug delivery devices of the '352, '711, and '255 patents are limited because each principally requires use of an optionally refillable powered pump to achieve prolonged and steady rates of drug delivery or do not disclose or suggest a mechanism for removal of old drug and providing new drug. Such pumps are expensive, require expensive approval processes, and are typically bulky and therefore uncomfortable and inconvenient for patient use. Without a pump to control release of the drug, delivery of solution-phase drug from a graft cuff-reservoir would typically decrease exponentially over a relatively short period of time.

Therefore, suitable refillable pumps are needed that deliver drug either through bulk movement of the drug vehicle (e.g., positive pressure, convection-based drug delivery) or that enable drug delivery by simple diffusion of drug from a drug reservoir under neutral pressure (diffusion-based drug delivery), and are optionally inexpensive, clinically approved, and patient appropriate.

In addition, while drug delivery devices exist that might be used for filling, refilling, emptying a graft drug reservoir (e.g., a cuff-reservoir), they lack suitable flow and pressure control resulting in volume expansion and contraction of the drug reservoir that can increase or decrease the diameter of the graft in contact with the drug reservoir. In addition, a lack of flow, pressure and volume control for filling, refilling or emptying a graft drug reservoir may result in forcing drug through the graft wall into the blood or “pulling” blood components back into the drug reservoir. A refillable drug delivery device that controls flow, pressure and reservoir volume during drug refill is also needed.

### SUMMARY

Aspects described herein provide refillable, controlled delivery devices for vascular delivery of drugs to blood vessels or to the luminal surface of a vascular graft.

In one aspect, a drug delivery device comprising a first layer and a drug depot comprising at least one drug is provided. In this aspect, when the drug depot is placed adjacent to a porous second layer, the drug is released from the drug depot in a controlled manner and diffuses across the second layer.

In another aspect, the first layer is a cuff (e.g., silicone cuff). In yet another aspect, the second layer is a microporous membrane. In a further aspect the drug depot comprises microbeads or microspheres containing at least one drug. In another aspect, the microbeads or microsphere comprise PLG poly (D,L-lactide)-co-glycolide. In yet another aspect, the drug can be selected from -olimus drugs (e.g., sirolimus, everolimus, zotarolimus, tacrolimus, pimecrolimus, temsirolimus, ridaforolimus or biolimus etc.).

Aspects described herein provide an implantable device for controlled drug delivery (e.g., prolonged or near-constant drug delivery) comprising an external casing, a first chamber having a first diaphragm, a second chamber having a second diaphragm and a needle stop, a dual-lumen needle, and a dual-lumen catheter. In this aspect, the dual-lumen needle comprises a drug-fill needle for filling the first chamber with new drug and a drug-withdrawal needle for removing old drug from the second chamber. Further aspects include a cuff drug reservoir providing a barrier that directs drug flow circumferentially when the reservoir is filled, refilled, or emptied.

In this aspect, the first diaphragm covers one side of the first chamber and second diaphragm covers one side of the second chamber. The first and second diaphragms are configured to be punctured by the drug-fill needle and drug-withdrawal needle respectively. The drug-fill needle can be configured to be longer than the drug-withdrawal needle in order for the drug-fill needle and drug withdrawal needle to simultaneously puncture the first and second diaphragms.

In this aspect, the dual-lumen catheter comprises a drug-fill lumen for conducting drug from the first chamber to a graft-cuff reservoir and a drug-withdrawal lumen for removing old drug from the graft-cuff reservoir to the second chamber.

### BRIEF DESCRIPTION OF THE DRAWINGS

FIG. 1 illustrates exemplary elements of the cuff-reservoir and dual-lumen implantable device;

FIG. 2 illustrates yet another alternative of the dual-lumen implantable device shown in FIG. 1;

FIG. 3 illustrates an example of refill and washout principles for a drug reservoir configured to deliver drug to a vascular graft in aspects described herein;

FIG. 4 shows a drug delivery device having a silicone cuff encasing a refillable drug reservoir that may contain drug microbeads. A microporous membrane may be optionally used to control drug delivery from a suitable drug formulation; and

FIG. 5 shows a drug delivery device having a silicone cuff encasing a refillable drug reservoir that may contain a controlled-release drug depot formulation. A microporous membrane may be optionally used to control drug delivery from a suitable drug formulation.

### DETAILED DESCRIPTION

Before describing exemplary aspects described herein, it is to be understood that the invention is not limited to the details of construction or process steps set forth in the following description. The aspects described herein are capable of being practiced or being carried out in various ways.

Aspects described herein provide drug delivery devices having an external casing, a first chamber having a first diaphragm, a second chamber having a second diaphragm and a needle stop, a dual-lumen needle, and a dual-lumen catheter. In one aspect, the dual-lumen needle comprises a needle drug-fill lumen and a needle drug-withdrawal lumen.

In a further aspect, the needle drug-fill lumen of the dual-lumen needle is configured to puncture the second diaphragm and operatively connect to the second chamber. In yet another aspect, the needle drug-withdrawal lumen of the dual-lumen needle is configured to puncture the first diaphragm and operatively connect to the first chamber.

The dual-lumen catheter can comprise a catheter drug-fill lumen and a catheter drug-withdrawal lumen. In this aspect, the catheter drug-fill lumen can be configured to operatively connect to the second chamber. In another aspect, the catheter drug-withdrawal lumen is configured to operatively connect to the first chamber. In a further aspect, the catheter drug-fill lumen and the catheter drug-withdrawal lumen can be configured to operatively connect to a drug reservoir.

The drug reservoir can further comprise a barrier configured to divide the drug reservoir into a first part and second part, wherein material in the first part can move to the second part in one direction. In this aspect, the catheter drug-fill lumen and the catheter drug-withdrawal lumen of the dual-lumen catheter are operatively connected to opposite sides of a drug reservoir formed by placing a barrier in the drug reservoir, wherein the barrier causes a material in the drug reservoir to move circumferentially when the reservoir is filled, refilled, or emptied.

The drug delivery devices can further comprise a cuff surrounding the drug reservoir and a graft surrounded by the drug reservoir. The cuff can be made of a material comprising silicone. The graft can be made of a material comprising ePTFE and can further comprise a rate-limiting membrane (e.g., microporous membrane). The rate-limiting membrane can be made of a material comprising at least one of polytetrafluoroethylene, polyvinylidinefluoride, polyethersulfone, polycarbonate, and cellulose esters.

In another aspect, drug reservoir contains a first drug (e.g., an olimus drug such as at least one of sirolimus, everolimus, zotarolimus, tacrolimus, pimecrolimus, temsirolimus, ridaforolimus or biolimus).

In another aspect, first drug can be selected from the group consisting of at least one of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, or jardiance.

In yet another aspect, the first drug can be selected from the group consisting of at least one of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

The drug reservoir can also contain a second drug in addition to the first drug.

In another aspect, the drug reservoir comprises a drug formulation (e.g., drug depot formulation, controlled release or extended release formulation, or a simple drug solution) containing the first drug. The controlled or extended release formulation can be selected from the group consisting of microbeads and microspheres.

The microbeads or micro spheres can comprise one or more materials selected from the group consisting of polyanhydride, PLG poly (D,L-lactide)-co-glycolide, poly DL lactide co-poly ethylene glycol (PELA) diblock copolymers, PLA-PEG-PLA triblocks, and PEG hydrophilic.

In another aspect, the second drug can be selected from the group consisting of at least one of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

In yet another aspect, the second drug can be selected from the group consisting of at least one of a chemotherapy drug, pain reliever, nutrient, or at least one agent to treat at least one of diabetes, arthritis, cancer, dehydration, or a migraine.

In a further aspect, the second drug can be selected from the group consisting of one or more of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

Aspects described herein provides method of filling a drug reservoir with new drug and withdrawing old drug from a drug reservoir by filling a drug reservoir with new drug and withdrawing old drug from a drug reservoir by infusing and withdrawing drug through a dual-lumen needle comprising a needle drug-fill lumen and a needle drug-withdrawal lumen wherein the needle drug-fill lumen is operatively connected to a first chamber and the needle drug-withdrawal lumen is operatively connected to a second chamber; filling the first chamber with new drug wherein the new drug flows through a catheter drug-fill lumen of a dual-lumen catheter operatively connected to the first chamber; filling the drug reservoir with new drug from the drug-fill lumen of the dual-lumen catheter wherein the old drug flows into a catheter drug-withdrawal lumen of the dual-lumen catheter, wherein the catheter drug-withdrawal lumen of the dual-lumen catheter is operatively connected to a second chamber; filling the second chamber with the old drug flowing through the catheter drug-withdrawal lumen of the dual-lumen catheter; and withdrawing the old drug from the second chamber through the needle drug-withdrawal lumen of the dual-lumen needle.

In one aspect, the drug reservoir is filled with new drug and old drug is withdrawn from the drug reservoir simultaneously.

In another aspect, the drug reservoir further comprises a barrier dividing the drug reservoir into a first part and second part, wherein new drug in the first part can move to the second part in one direction.

In one aspect, the first chamber further comprises a first diaphragm and the second chamber further comprises a second diaphragm.

In another aspect, the needle drug-withdrawal lumen of the dual-lumen needle punctures the first diaphragm. In yet another aspect, the needle drug-fill lumen of the dual-lumen needle punctures the second diaphragm.

In yet another aspect, new drug comprises a first drug (e.g., an olimus drug selected from the group consisting of at least one of sirolimus, everolimus, zotarolimus, tacrolimus, pimecrolimus, temsirolimus, ridaforolimus or biolimus).

In one aspect, the first drug is selected from the group consisting of at least one of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

In a further aspect, the first drug is selected from the group consisting of at least one of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

In another aspect, the new drug further comprises a second drug.

In another aspect, the drug reservoir further comprises a drug formulation (e.g., a drug depot formulation, a controlled or an extended release formulation, or a simple drug solution) containing the first drug.

The controlled or extended release formulation can be selected from the group consisting of microbeads and microspheres. The microbeads or microspheres can comprise one or more materials selected from the group consisting of polyanhydride, PLG poly (D,L-lactide)-co-glycolide, poly DL lactide co-poly ethylene glycol (PELA) diblock copolymers, PLA-PEG-PLA triblocks, and PEG hydrophilic.

In a further aspect, the second drug is selected from the group consisting of at least one of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

In another aspect, the second drug is selected from the group consisting of at least one of a chemotherapy drug, pain reliever, nutrient, or at least one agent to treat at least one of diabetes, arthritis, cancer, dehydration, or a migraine.

In a further aspect, the second drug can be selected from the group consisting of at least one of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

Optionally, an infusion/withdrawal pump can be operationally connected to the dual-lumen needle to actuate the needle.

The term “operatively connect,” in the context of aspects described herein, refers to operating an aspect or element of a device from one aspect or element of the device to or through another element or aspect of the device. For example, a dual-lumen needle as described herein can puncture the first diaphragm, enter the first chamber, puncture the second diaphragm and enter the second chamber.

The term “old drug” refers to a carrier that contains expired drug or formerly contained drug. The term “new drug” refers to a carrier containing unexpired drug, replacement drug, or a new drug to be re-supplied to a drug reservoir. The term “drug fluid” refers to any suitable fluid for use in a human or animal that contains a drug or combination of drugs. Drug fluid can also be depleted or removed from the drug fluid through delivery, for example, to bodily fluids or tissues.

The terms “simultaneous” or “simultaneously” refer to events occurring at nearly or close to the same time and do not require the events occur at exactly the same time.

The term “extended release” refers to substantially uniform rate of release of a drug over a period of time (e.g., having a rate diminishing by no more than about 50% for approximately 30 days) conducive for treatment of a condition. The rate of release can be nearly constant, substantially constant, prolonged-release or controlled-release and does not require that the rate remain exactly the same (zero-order kinetics) over a defined period of time.

The term “reservoir” refers to an article or device capable of holding or retaining fluid.

Previously described devices can be used to deliver drug from a drug depot encased in a silicone cuff and placed adjacent to a vascular graft (see '352 patent). However, the uniformity of drug delivery through the lumenal surface of certain porous graft materials may be poorly controlled (e.g., circumferentially non-uniform).

The '711 patent refers to use of a microporous membrane to provide uniform drug delivery. However, once the cuff drug reservoir has been depleted of the provided drug, the cuff drug reservoir must be refilled or another cuff having more drug must be implanted. Moreover, as discussed herein, the microporous membrane of the '711 patent does not control the rate of drug delivery as described herein, but only the uniformity of drug delivery through a porous graft surface. While the '255 patent refers to removable and replaceable cuffs, this procedure is time consuming, expensive, and inconvenient for the patient.

Aspects described herein provide extended-release kinetics using, for example, passive drug diffusion from either microbeads or other controlled-release depot formulations into the reservoir space, with subsequent drug diffusion across a porous graft wall. Optionally, the diffusion rate can be further controlled, if necessary, by a microporous membrane. In another aspect, near-constant or prolonged drug delivery can be achieved until the microbeads or depot-vehicle is depleted.

The terms “microbeads” and “microspheres” refer to solid elements containing one or more drugs that are released by diffusion out of the solid (e.g., leaving the solid behind), or by dissolution of the solid. In one aspect, microbeads are of sufficient size not to cross a macroporous membrane (e.g., ePTFE graft wall) or a microporous membrane, when one is used.

The term “drug depot formulation” refers to formulations of drug(s) combined with a carrier (e.g., vehicle) that cannot substantially cross a macro or microporous membrane. In this aspect, the drug can be released by diffusion out of the carrier vehicle or upon vehicle dissolution. Vehicles used in drug depot formulations include particulates, colloids, heterogeneous suspensions, and polymer or biochemical molecules, complexes or aggregates having sufficient size, shape, molecular weight, or other properties that substantially prevent the vehicle from crossing the macro/microporous membrane in significant amounts (e.g., vs. the amount of free drug that can diffuse across the membrane). Other mechanisms for substantially preventing a vehicle carrier from crossing a porous membrane include electrostatic, ionic, hydrophobic, hydrophilic, and magnetic properties.

The properties of drug depot formulations, drug microbeads, and drug microspheres are different from and more complex than drugs dissolved in a simple solution because the properties of the vehicle and the interaction between the vehicle and drug can significantly affect the diffusion properties of the drug component of drug depot formulations, drug microbeads, and drug microspheres.

Aspects described herein provide a drug formulation (e.g., drug depot formulation or controlled-release formulation) for achieving extended drug delivery. The same considerations described herein for use of microbeads or microspheres apply to the use of an optional microporous membrane to limit drug diffusion. If the drug formulation is a simple drug solution containing a solubilized drug that could easily cross the ePTFE graft pores, unlike microbeads, then the microporous membrane can optionally serve to limit the movement (by diffusion or convection) of drug across the graft wall and into the blood. The properties of microporous membranes are described in the literature. For example, microporous membranes marketed by Millipore Sigma, Inc. (Burlington, Mass.) include membranes based on polytetrafluoroethylene, polyvinylidinefluoride, polyethersulfone, polycarbonate, and cellulose esters.

For example, if a dissolved drug solution is loaded into the reservoir, microporous membrane with a pore size from about 1 nm (1 nanometer) to about 1 μm (1 micrometer) can be used.

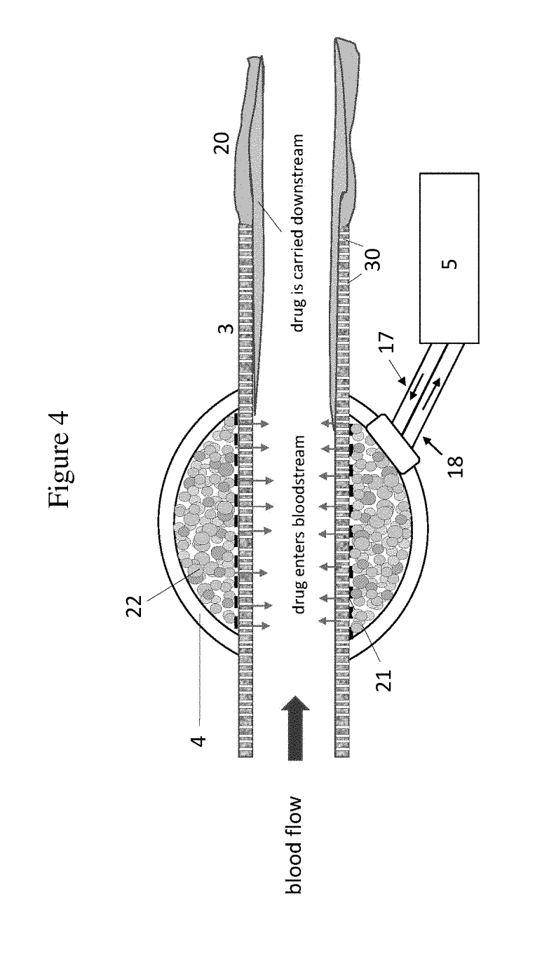

In one aspect, drug-containing microbeads can provide extended drug release into the reservoir space (see, e.g., FIG. 4).

Exemplary drug-containing microbeads include, but are not limited to:

(1) Surface eroding polymers like polyanhydride (e.g., composed of hydrophobic monomers linked by liable bonds). In one aspect, these polymers keep water out of polymer bulk but break down to oligomers and monomers via hydrolysis with first-order kinetics. Drug release is independent of polymer molecular weight (Reference 5).

(2) Bulk eroding polymers like PLG poly (D,L-lactide)-co-glycolide readily allow water permeation and polymer degradation throughout the matrix. While these polymers can be characterized by early and undesirable “burst” effects of drug release, drug release is then near zero-order (Reference 7).

Further, it has been demonstrated that the pH of the PLGA microenvironment can affect the drug release rate and degradation rate of the polymer. Lowering the pH from 7.4 to 2.4 accelerated the release kinetics of the microsphere preparation (Reference 8).

(3) Other PEG+PLG=poly DL lactide co-poly ethylene glycol (PELA) diblock copolymers.

(4) PLA-PEG-PLA triblocks.

(5) PLG-PEG-PLG triblocks.

(6) PEG hydrophilic, better entrapment (Reference 5).

Drug release increases with decreasing particle size. (Reference 5). Since ePTFE pore size at least 30 μM, in one aspect the microsphere or microbead particles can be greater than 30 μM to about 500 μM.

BSA or mannitol can be added as stabilizers to the microbeads or microspheres (Reference 5).

Drugs that can be added to a drug depot or used in microbeads and microspheres as described herein include, but are not limited to, the pharmaceutical formulation wherein the olimus drug includes at least one of sirolimus, everolimus, zotarolimus, tacrolimus, pimecrolimus, temsirolimus, ridaforolimus or biolimus.

Additional drugs that can be used alone or in combination with other drugs include at least one of the following drugs: actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, or jardiance; at least one chemotherapy drug; at least one pain reliever; at least one nutrient; or at least one agent to treat at least one of diabetes, arthritis, cancer, dehydration, or a migraine.

Additional classes of drugs that can used in aspects described herein include, but are not limited to antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, ribonucleases and any combinations thereof.

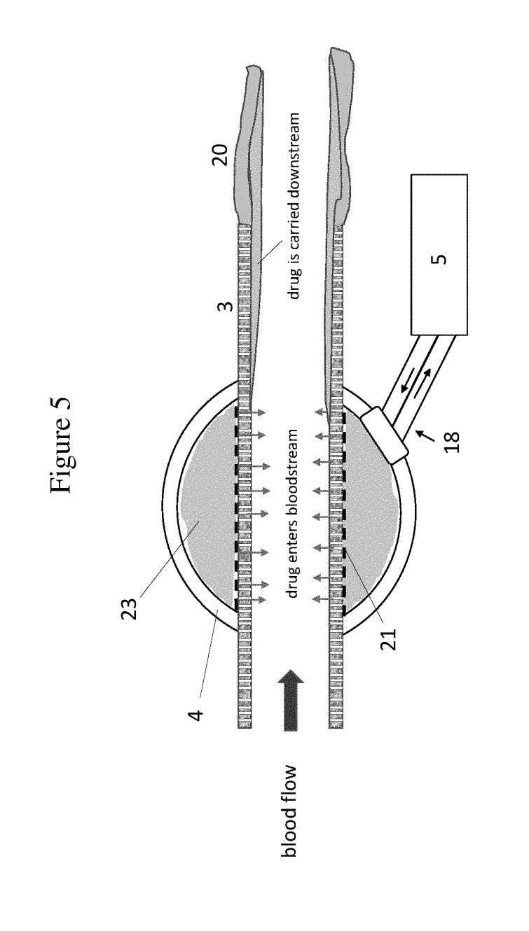

In this aspect, if the microbeads, microspheres or drug vehicle used in drug depot formulations cannot cross the ePTFE (or other) graft wall (e.g., having a nominal porosity of at least 30 microns), then there is no need for an additional microporous membrane to prevent the beads from crossing the graft wall and entering the blood. However, if drug is released too quickly from the beads or drug depot vehicle, a microporous membrane may be added, overlaying the external surface of the ePTFE graft, that limits diffusion to the graft external surface and subsequently through the graft wall and into the blood stream (FIGS. 4, 5). Thus, a purpose of the microporous membrane can be controlling the rate of diffusion of drug out of the reservoir.

The use of a microporous membrane, similarly placed in the cuff-reservoir, to control the uniformity of drug delivery through the graft wall and into the blood has been described ('711 patent). However, the '711 patent describes the microporous membrane for controlling the uniformity of drug delivery only, without affecting the overall rate of drug delivery. Since drug delivery in the '711 patent is from positive pressure convection-driven pumping of drug-containing solution, the '711 patent does not describe the use of a diffusion-controlling membrane.

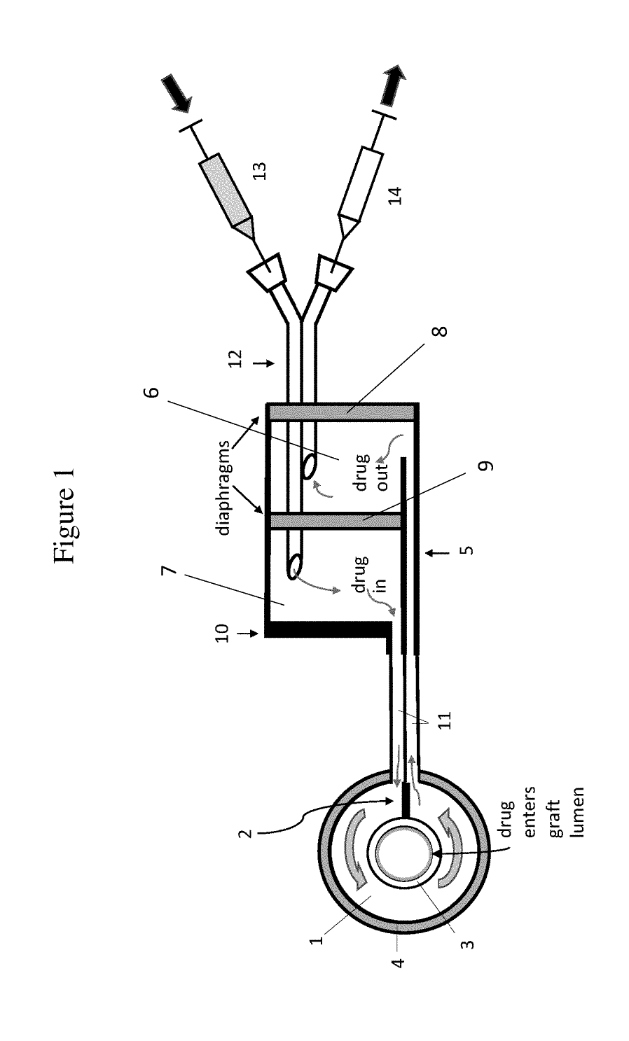

With reference to FIG. 1, an exemplary aspect is shown having drug reservoir 1, barrier 2, ePTFE graft 3, and silicone cuff 4, wherein barrier 2 can be used to direct a circumferential washout drug flow when filling, refilling, or emptying drug reservoir 1. Dual chamber device 5 is shown having first chamber 6, second chamber 7, first diaphragm 8, second diaphragm 9, and needle stop 10. Dual-lumen catheter 11, dual-lumen needle 12, fill syringe 13, and withdrawal syringe 14 are also shown.

In this aspect, drug is provided to dual chamber device 5 with fill syringe 13 through dual-lumen needle 12 into second chamber 7 and to one side of drug reservoir 1 as delineated by barrier 2, through dual-lumen catheter 11. Barrier 2 forces circumferential washout flow around drug reservoir 1 as shown. Old drug is pushed around drug reservoir 1 to the other side delineated by barrier 2 and out through the dual-lumen catheter 11 to first chamber 6 where it can be withdrawn through dual-lumen needle 12 and into withdrawal syringe 14.

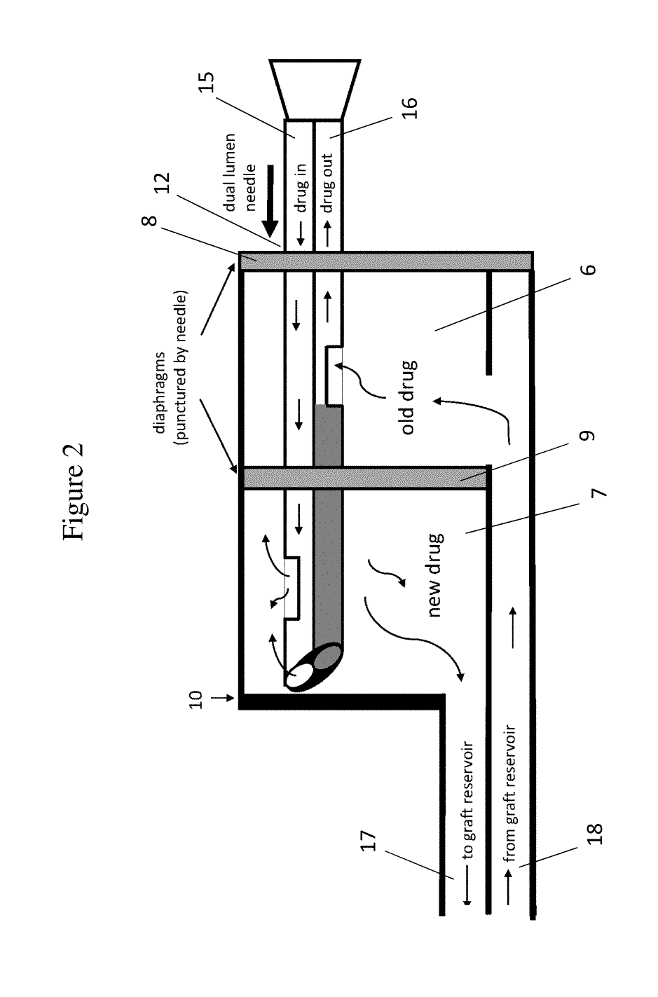

FIG. 2 shows a cross-section close up view of dual chamber device 5 in the exemplary device of FIG. 1. Dual-lumen needle 12 having a drug-in needle portion 15 and a drug-out needle portion 16 is shown inserted through first diaphragm 8 and second diaphragm 9 and through first chamber 6 and into second chamber 7, prior to needle stop 10. In this aspect, new drug is injected using dual-lumen needle 12 through drug-in needle portion 15, and released in second chamber 7, where it flows to ePTFE graft 3 (not shown) through drug-in catheter portion 17. Drug-out needle portion 16 is plugged from the section inserted in first chamber 6 through the section in second chamber 7 to prevent old drug from flowing into second chamber 7. Old drug returns through drug-out catheter portion 18 into first chamber 6 into drug-out needle portion 16 and is withdrawn using dual-lumen needle 12 and withdrawal syringe 14 (not shown). Dual-lumen needle 12 is prevented from puncturing the distal portion of dual chamber device 5 by needle stop 10.

In this aspect, drug can be provided to dual chamber device 5 by injecting drug with fill syringe 13 which has penetrated first diaphragm 8 and second diaphragm 9 through first chamber 6 and into second chamber 7, prior to needle stop 10. Second chamber 7 is fluidly connected to drug reservoir 1 through the drug-in catheter portion 17 of dual-lumen catheter 11, between ePTFE graft 3 and silicone cuff 4 on one side of barrier 2. As shown, drug is directed circumferentially around drug reservoir 1 where it can be delivered into the lumen as described herein.

Depleted drug fluid can be withdrawn by pulling on withdrawal syringe 14 to draw drug out of drug reservoir 1, through the drug out catheter portion 18 of dual-lumen catheter 11, into first chamber 6 and out through drug out needle portion 16 into drug withdrawal syringe 14.

In one aspect, needle stop 10 is the end of the device that may be contacted by the needle in some embodiments. In another aspect, the needle can be configured so that the needle hub contacting the first diaphragm limits needle penetration depth and can be made of materials that are biocompatible and resistant to breakage, tearing or degradation both inside and outside the body (e.g., polycarbonate, PVC, polyurethane; coated plastic; metal or ceramic, coated metal or ceramic, etc.).

First diaphragm 8 and second diaphragm 9 can be made from a variety of suitable materials. See, e.g., Reference 4, below.

Dual-lumen needle 12 can be of any material and configuration for puncturing first and second diaphragm. For example, a 21 gauge needle (0.82 mm o.d.×0.51 mm i.d.) can fit inside a 16 gauge needle (1.65 mm o.d.×1.19 mm i.d.). In one aspect, the outside diameter of dual-lumen needle 12 can therefore be about 2.0 mm or less. The end of the device that will punctured, typically a circular diaphragm, can be small, with a diameter of about 5 mm or any suitable configuration (e.g., as shown in FIGS. 1-2).

The external casing of dual chamber device 5 can be made of materials that are biocompatible and resistant to breakage, tearing or degradation both inside and outside the body (e.g., polycarbonate, PVC, polyurethane; coated plastic; metal or ceramic, coated metal or ceramic, etc.).

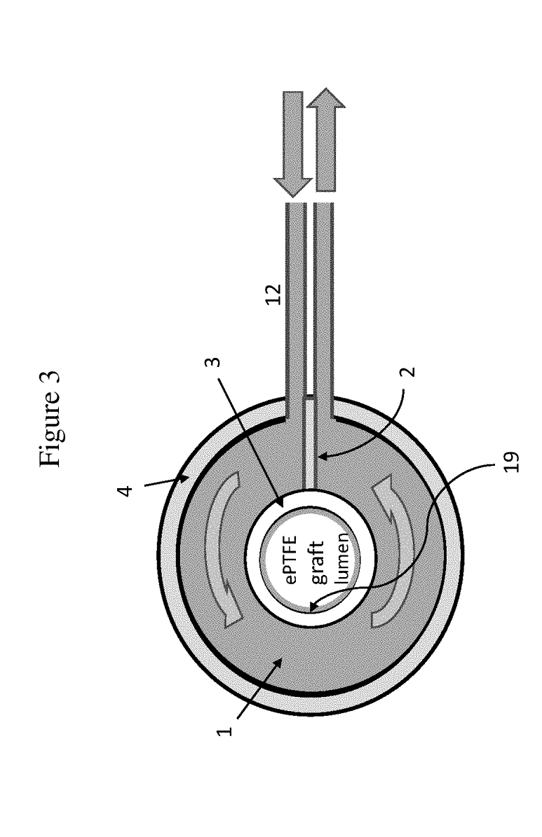

FIG. 3 shows a cross-section close-up of an exemplary ePTFE graft 3 and silicone cuff 4 in the exemplary device of FIG. 1. Drug reservoir 1 is shown with drug flowing circumferentially between silicone cuff 4 and ePTFE graft 3, with drug entering and exiting through dual-lumen catheter 12 around barrier 2. Drug boundary layer 19 is shown on the luminal surface of ePTFE graft 3.

With reference to FIG. 4, an exemplary drug reservoir and cuff device is shown in cross-section with ePTFE (expanded polytetrafluoroethylene) graft 3 connected to vein 20. ePTFE graft 3 has rate limiting membrane 21. Silicone cuff 4 is shown surrounding ePTFE graft 3 and containing, for example, drug microspheres 22. Barrier 2 (not shown) directs circumferential flow of drug as described herein with respect to FIGS. 1 and 3. In this aspect, drug microspheres 22 contain suitable drug(s) and surface-eroding polymer(s), as described herein, to release the drug at a desired rate. The drug can enter the bloodstream through exemplary pores in the ePTFE graft 3. The drug in the reservoir is electively filled, removed and replaced using dual chamber device 5 as illustrated, for example, in FIGS. 1-2.

With reference to FIG. 5, an exemplary device is shown in cross-section with ePTFE (expanded polytetrafluoroethylene) graft 3 connected to vein 20. ePTFE graft 3 has rate limiting membrane 21. Silicone cuff 4 is shown surrounding ePTFE graft 3 and containing, for example, drug-depot formulation 23. In this aspect, the drug-depot formulation 23 contains suitable drug(s) and surface-eroding polymer(s), as described herein, to release the drug at a desired rate. The drug can enter the bloodstream through exemplary pores in the ePTFE graft 3.

The drug in the reservoir is electively filled, removed and replaced using dual chamber device 5 as illustrated, for example, in FIGS. 1-3.

It is understood that the graft and cuff can be made of any suitable material (e.g., and the graft can be affixed or attached to the cuff using any suitable adhesive or by any suitable manner to ensure that the drug fluid does not substantially leak. See, e.g., '352, '711, and '255 patents.

An exemplary dual-lumen catheter, or two separate catheter lines, can be used to connect the refill device to the reservoir. As shown in FIG. 2, this aspect can be used as a dual-chamber infusion-withdrawal device. Elements used to make such catheters are known in the art (See, e.g., References 1-3). In one aspect, this device can be implanted and made accessible under the skin and therefore can be made to be small (e.g., 2 mm-2 cm thick). In one aspect, this device can be a round or flattened cylinder, or any suitable shape.

### REFERENCES

(1) U.S. Pat. No. 5,399,352
(2) U.S. Pat. No. 8,808,255
(3) U.S. Pat. No. 8,721,711
(4) U.S. Pat. No. 6,544,246
(5) Kim K and Pack DW. Microspheres for Drug Delivery. BioMEMS and Biomedical Nanotechnology, Vol. 1: Biological and Biomedical Nanotechnology, Editors (Ferrari, Lee, Lee). 2006. p. 520.
(6) Rajathurai et al. Periadventitial Rapamycin-Eluting Microbeads Promote Vein Graft Disease in Long-Term Pig Vein-Into-Artery Interposition Grafts. Circ Cardiovasc Interv. 2010; 3:157-165.
(7) Zheng-Xing Su, Ya-Nan Shi, Le-Sheng Teng, Xiang Li, Le-xi Wang, Qing-Fan Meng, Li-Rong Teng & You-Xin Li. (2011). Biodegradable poly(D, L-lactide-co-glycolide) (PLGA) microspheres for sustained release of risperidone: Zero-order release formulation, Pharmaceutical Development and Technology, 16:4, 377-384, DOI: 10.3109/10837451003739297.
(8) Zolnik Banu S and Burgess Diane J. (2007). Effect of acidic pH on PLGA microsphere degradation and release. J Controlled Release 122(3):338-344.
(9) http://www.emdmillipore.com/US/en/life-science-research/chromatography-sample-preparation/microfiltration-membranes/FqCb.qB 7ZkAAAFBDQZ1vzJE,nav

What is claimed is:

1. A drug delivery device comprising an external casing, a first chamber having a first diaphragm, a second chamber having a second diaphragm and a needle stop, a dual-lumen needle, and a dual-lumen catheter.

2. The device of claim 1, wherein dual-lumen needle comprises a needle drug-fill lumen and a needle drug-withdrawal lumen.

3. The device of claim 2, wherein the needle drug-fill lumen of the dual-lumen needle is configured to puncture the second diaphragm and operatively connect to the second chamber.

4. The device of claim 2, wherein the needle drug-withdrawal lumen of the dual-lumen needle is configured to puncture the first diaphragm and operatively connect to the first chamber.

5. The device of claim 1, wherein the dual-lumen catheter comprises a catheter drug-fill lumen and a catheter drug-withdrawal lumen.

6. The device of claim 5, wherein the catheter drug-fill lumen is configured to operatively connect to the second chamber.

7. The device of claim 5, wherein the catheter drug-withdrawal lumen is configured to operatively connect to the first chamber.

8. The device of claim 5, wherein the catheter drug-fill lumen and the catheter drug-withdrawal lumen are configured to operatively connect to a drug reservoir.

9. The device of claim 8, wherein the drug reservoir further comprises a barrier configured to divide the drug reservoir into a first part and second part, wherein material in the first part can move to the second part in one direction.

10. The device of claim 9, wherein the catheter drug-fill lumen and the catheter drug-withdrawal lumen of the dual-lumen catheter are operatively connected to opposite sides of a drug reservoir formed by placing a barrier in the drug reservoir, wherein the barrier causes a material in the drug reservoir to move circumferentially when the drug reservoir is filled, refilled, or emptied.

11. The device of claim 10, further comprising a cuff surrounding the drug reservoir and a graft surrounded by the drug reservoir.

12. The device of claim 11, wherein the cuff is made of a material comprising silicone.

13. The device of claim 11, wherein the graft is made of a material comprising ePTFE.

14. The device of claim 11, wherein the graft further comprises a rate-limiting membrane.

15. The device of claim 14, wherein the rate-limiting membrane is made of a material comprising at least one of polytetrafluoroethylene, polyvinylidinefluoride, polyethersulfone, polycarbonate, and cellulose esters.

16. The device of claim 11, wherein the drug reservoir contains a first drug.

17. The device of claim 16, wherein the first drug is an olimus drug.

18. The device of claim 16, wherein the first drug is selected from the group consisting of at least one of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, or jardiance.

19. The device of claim 16, wherein the first drug is selected from the group consisting of at least one of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

20. The device of claim 17, wherein the olimus drug is selected from the group consisting of at least one of sirolimus, everolimus, zotarolimus, tacrolimus, pimecrolimus, temsirolimus, ridaforolimus or biolimus.

21. The device of claim 16, wherein the drug reservoir contains a second drug.

22. The device of claim 16, further comprising a drug formulation containing the first drug.

23. The device of claim 22, wherein the drug formulation is selected from the group consisting of a drug depot formulation, a controlled release formulation, an extended release formulation, and a simple drug solution.

24. The device of claim 23, wherein the controlled release formulation is selected from the group consisting of microbeads and microspheres.

25. The device of claim 24, wherein the microbeads or microspheres comprise one or more materials selected from the group consisting of polyanhydride, PLG poly (D,L-lactide)-co-glycolide, poly DL lactide co-poly ethylene glycol (PELA) diblock copolymers, PLA-PEG-PLA triblocks, and PEG hydrophilic.

26. The device of claim 21, wherein the second drug is selected from the group consisting of one or more of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

27. The device of claim 26, wherein the second drug is selected from the group consisting of at least one a chemotherapy drug, pain reliever, nutrient, or at least one agent to treat at least one of diabetes, arthritis, cancer, dehydration, or a migraine.

28. The device of claim 26, wherein the second drug is selected from the group consisting of at least one of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

29. A method of filling a drug reservoir with new drug and withdrawing old drug from a drug reservoir comprising:
filling a drug reservoir with new drug and withdrawing old drug from a drug reservoir by infusing and withdrawing drug through a dual-lumen needle comprising a needle drug-fill lumen and a needle drug-withdrawal lumen wherein the needle drug-fill lumen is operatively connected to a first chamber and the needle drug-withdrawal lumen is operatively connected to a second chamber;
filling the first chamber with new drug wherein the new drug flows through a catheter drug-fill lumen of a dual-lumen catheter operatively connected to the first chamber;
filling the drug reservoir with new drug from the catheter drug-fill lumen of the dual-lumen catheter wherein the old drug flows into a catheter drug-withdrawal lumen of the dual-lumen catheter, wherein the catheter drug-withdrawal lumen of the dual-lumen catheter is operatively connected to a second chamber;
filling the second chamber with the old drug flowing through the catheter drug-withdrawal lumen of the dual-lumen catheter; and
withdrawing the old drug from the second chamber through the needle drug-withdrawal lumen of the dual-lumen needle.

30. The method of claim 29, wherein the drug reservoir is filled with new drug and old drug is withdrawn from the drug reservoir simultaneously.

31. The method of claim 29, wherein the drug reservoir further comprises a barrier dividing the drug reservoir into a first part and second part, wherein new drug in the first part can move to the second part in one direction.

32. The method of claim 29, wherein the first chamber further comprises a first diaphragm and the second chamber further comprises a second diaphragm.

33. The method of claim 32, wherein the needle drug-withdrawal lumen of the dual-lumen needle punctures the first diaphragm.

34. The method of claim 32, wherein the needle drug-fill lumen of the dual-lumen needle punctures the second diaphragm.

35. The method of claim 29, wherein the new drug comprises a first drug.

36. The method of claim 35, wherein the first drug is an olimus drug.

37. The method of claim 35, wherein the first drug is selected from the group consisting of one or more of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

38. The method of claim 35, wherein the first drug is selected from the group consisting of one or more of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.

39. The method of claim 36, wherein the olimus drug is selected from the group consisting of at least one of sirolimus, everolimus, zotarolimus, tacrolimus, pimecrolimus, temsirolimus, ridaforolimus or biolimus.

40. The method of claim 35, wherein the new drug further comprises a second drug.

41. The method of claim 35, further comprising a drug formulation containing the first drug.

42. The method of claim 41, wherein the drug formulation is selected from the group consisting of a drug depot formulation, a controlled release formulation, an extended release formulation, and a simple drug solution.

43. The method of claim 42, wherein the controlled release formulation is selected from the group consisting of microbeads and microspheres.

44. The method of claim 43, wherein the microbeads or microspheres comprise one or more materials selected from the group consisting of polyanhydride, PLG poly (D,L-lactide)-co-glycolide, poly DL lactide co-poly ethylene glycol (PELA) diblock copolymers, PLA-PEG-PLA triblocks, and PEG hydrophilic.

45. The method of claim 40, wherein the second drug is selected from the group consisting of actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bedamustine, panitumumab, bortezomib, venofer, zoledronic acid, thiazolidinediones, glipizide, glimepiride, metformin, victoza, and jardiance.

46. The method of claim 40, wherein the second drug is selected from the group consisting of at least one chemotherapy drug, pain reliever, nutrient, or at least one agent to treat at least one of diabetes, arthritis, cancer, dehydration, or a migraine.

47. The method of claim 40, wherein the second drug is selected from the group consisting of antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, and ribonucleases.
